(19) United States
(12) Patent Application Publication
(10) Pub. No.: US 2009/0247854 A1
(43) Pub. Date: Oct. 1, 2009

(54) Retractable Sensor Cable For A Pulse Oximeter

(75) Inventors: Daryl L. Bordon, Livemore, CA (US); Brian R. Ackley, Pleasanton, CA (US); Robin S. Boyce, Pleasanton, CA (US); William J. Durban, Pleasanton, CA (US); Sandra Jones, Auburn, CA (US); Gina To, San Ramon, CA (US); Steven J. Wong, Santa Clara, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/412,990

(22) Filed: Mar. 27, 2009

Related U.S. Application Data
(60) Provisional application No. 61/072,097, filed on Mar. 27, 2008.

(51) Int. Cl. A61B 5/1455

(52) U.S. Cl. 600/344

(57) ABSTRACT

Provided is a method and apparatus for storing of a sensor cable used with a medical device. The medical device may include a retraction device that is activated by depressing a lever. Once the lever is depressed, the sensor cable may automatically wind itself around a spool inside of the medical device. Additionally, an automatic stop feature prevents a sensor cable from retracting without depression of the lever, thus maintaining the exact length of cable required to connect a monitor to the monitoring site on a patient. The retraction of the sensor cable may allow for storage of the cable in the monitor itself, or may allow for storage of the cable into the retraction device, which may be detachable from the monitor.

### RELATED APPLICATIONS

This application claims priority to U.S. Provisional Application No. 61/072097, filed Mar. 27, 2008, and is incorporated herein by reference in its entirety.

### BACKGROUND

The present disclosure relates generally to medical devices and, more particularly, to the storage of components utilized in conjunction with the medical devices.

This section is intended to introduce the reader to various aspects of art that may be related to various aspects that are described and/or claimed below. This discussion is believed to be helpful in providing the reader with background information to facilitate a better understanding of these various aspects. Accordingly, it should be understood that these statements are to be read in this light, and not as admissions of prior art.

In the field of medicine, there is a need to monitor physiological characteristics of a patient. Accordingly, a wide variety of devices and techniques have been developed for monitoring the physiological characteristics of a patient. One such technique for monitoring certain physiological characteristics of a patient (e.g., blood flow characteristics) is commonly referred to as pulse oximetiy. Devices which perform pulse oximetry are commonly referred to as pulse oximeters. Pulse oximeters may be used to measure physiological characteristics such as the blood-oxygen saturation of hemoglobin in arterial blood, the volume of individual blood pulsations supplying the tissue, and/or the rate of blood pulsations corresponding to each heartbeat of a patient.

Specifically, these measurements may be acquired using a non-invasive sensor that transmits electromagnetic radiation, such as light, through a patient's tissue and that photoelectrically detects the absorption and/or scattering of the transmitted light in such tissue. Physiological characteristics may then be calculated based upon the amount of light absorbed and/or scattered. More specifically, the light passed through the tissue may be selected to be of one or more wavelengths that may be absorbed and/or scattered by the blood in an amount correlative to the amount of blood constituent present in the tissue. The measured amount of light absorbed and/or scattered may then be used to estimate the amount of blood constituent in the tissue using various algorithms.

The non-invasive sensor described above typically is connected to a pulse oximeter monitor via a cable. However, the cables are typically fixed in length. This may be problematic because more or less cable than is provided may be required for monitoring physiological characteristics of a patient. For example, if the fixed length of the sensor cable is longer than required to reach a patient for monitoring, the remaining cable may become problematic since the remaining length of sensor cable tends to dangle from the monitor where it may become twisted with other cables, for example. When the sensor cable is too short to reach the patient, fixed length extensions are typically used, often leading to an excess of cable with the similar problems discussed above.

Furthermore, when the pulse oximeter is not in use, the sensor cables must be stored, and there may not be a convenient location to store the cables. One solution has been to wrap the cables around the monitor, but this may damage the cables and shorten their lifespan. A second solution is to store the cables independently of the monitor. However, valuable time may be lost while searching for the separately stored sensor cables.

### SUMMARY

Certain aspects commensurate in scope with the originally claimed subject matter are set forth below. It should be understood that these aspects are presented merely to provide the reader with a brief summary of certain embodiments and that these aspects are not intended to limit the scope of the claims. Indeed, the claims may encompass a variety of aspects that may not be set forth below.

In accordance with one embodiment, there is provided a pulse oximeter that includes a retraction device. During monitoring, the retraction device allows a user to expose the length of cable appropriate to connect a monitor to the monitoring site on a patient. The retraction device also will maintain the selected length of cable exposed during monitoring. The retraction device further allows for retraction of the sensor cord into the pulse oximeter for ease of storage.

### BRIEF DESCRIPTION OF THE DRAWINGS

Certain embodiments may be understood reading the following detailed description and upon reference to the drawings in which:

FIG. 1 illustrates a simplified block diagram of a pulse oximeter in accordance with an embodiment;

FIG. 2 illustrates a front view of a pulse oximeter in accordance with an embodiment;

FIG. 2A illustrates a side view of the sensor mechanism illustrated in FIG. 2;

FIG. 2B illustrates a top view of the sensor mechanism illustrated in FIG. 2;

FIG. 2C illustrates a more detailed front view of a retraction housing portion of the pulse oximeter illustrated in FIG. 2;

FIG. 3 illustrates a side view of an embodiment the retraction mechanism in the pulse oximeter illustrated in FIG. 2;

FIG. 4 illustrates a front view of the retraction mechanism illustrated in FIG. 3;

FIG. 4A illustrates a detailed view the electrical connection system of the retraction mechanism illustrated in FIG. 4;

FIG. 5 illustrates a top view of the retraction mechanism illustrated in FIG. 3;

FIG. 6 illustrates a perspective view of a portion of the retraction mechanism illustrated in FIG. 5;

FIG. 7 an exploded view of the retraction mechanism illustrated in FIG. 5; and

FIG. 8 illustrates a front view of a pulse oximeter and retraction mechanism in accordance with another embodiment.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

The present disclosure is directed to a retraction device for use with a pulse oximeter or other suitable medical devices. During monitoring, the retraction device allows a user to expose, and maintain, the exact length of cable appropriate to connect a patient to a pulse oximeter monitor. Upon completion of a monitoring session, the retraction device allows for retraction of the sensor cable for ease of storage.

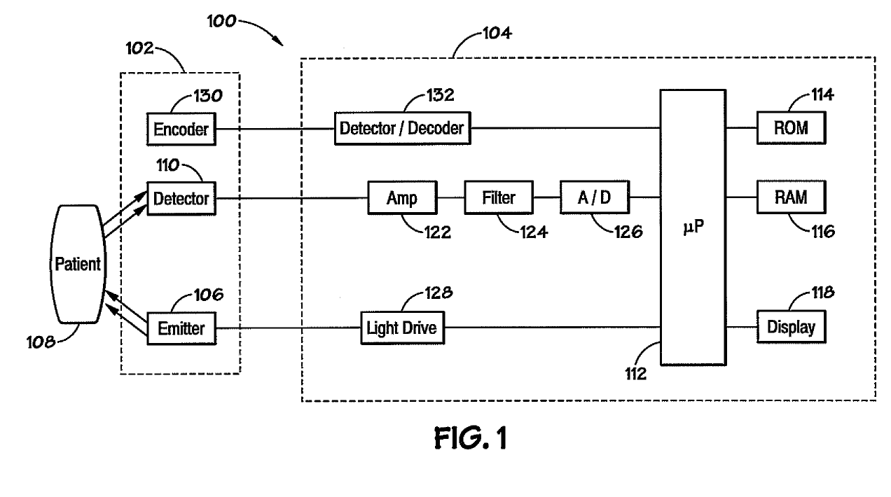

Turning to FIG. 1, a simplified block diagram of a medical device is illustrated in accordance with an embodiment. The medical device may be a pulse oximeter 100. The pulse oximeter 100 may include a sensor 102 having one or more emitters 106 configured to transmit electromagnetic radiation, i.e., light, into the tissue of a patient 108. For example, the emitter 106 may include a plurality of LEDs operating at discrete wavelengths, such as in the red and infrared portions of the electromagnetic radiation spectrum. Alternatively, the emitter 106 may be a broad spectrum emitter, or it may include wavelengths for measuring water fractions.

The sensor 102 may also include one or more detectors 110. The detector 110 may be a photoelectric detector which may detect the scattered and/or reflected light from the patient 108. Based on the detected light, the detector 110 may generate an electrical signal, e.g., current, at a level corresponding to the detected light. The sensor 102 may direct the electrical signal to the monitor 104 for processing and calculation of physiological parameters.

In this embodiment, the monitor 104 is a pulse oximeter, such as those available from Nellcor Puritan Bennett L.L.C. The monitor 104 may include an amplifier 122 and a filter 124 for amplifying and filtering the electrical signals from the sensor 102 before digitizing the electrical signals in the analog-to-digital converter 126. Once digitized, the signals may be used to calculate the physiological parameters of the patient 108. The monitor 104 may also include one or more processors 112 configured to calculate physiological parameters based on the digitized signals from the analog-to-digital converter 126 and further using algorithms programmed into the monitor 104. The processor 112 may be connected to other component parts of the monitor 104, such as one or more read only memories (ROM) 114, one or more random access memories (RAM) 116, and a display 118. The ROM 410 and the RAM 412 may be used in conjunction, or independently) to store the algorithms used by the processors in computing physiological parameters. The ROM 114 and the RAM 116 may also be used in conjunction, or independently, to store the values detected by the detector 110 for use in the calculation of the aforementioned algorithms.

Further, the monitor 104 may include a light drive unit 128. Light drive unit 128 may be used to control timing of the emitter 106. An encoder 130 and decoder 132 may be used to calibrate the monitor 104 to the actual wavelengths being used by the emitter 106. The encoder 130 may be a resistor, for example, whose value corresponds to the actual wavelengths and to coefficients used in algorithms for computing the physiological parameters. Alternatively, the encoder 130 may be a memory device, such as an EPROM, that stores wavelength information and/or the corresponding coefficients. For example, the encoder 130 may be a memory device such as those found in OxiMax® sensors available from Nellcor Puritan Bennett L.L.C. The encoder 130 may be communicatively coupled to the monitor 104 in order to communicate wavelength information to the decoder 132. The decoder 132 is provided for receiving and decoding the wavelength information from the encoder 130. Once decoded, the information is transmitted to the processor 112 for utilization in calculation of the physiological parameters of the patient 108.

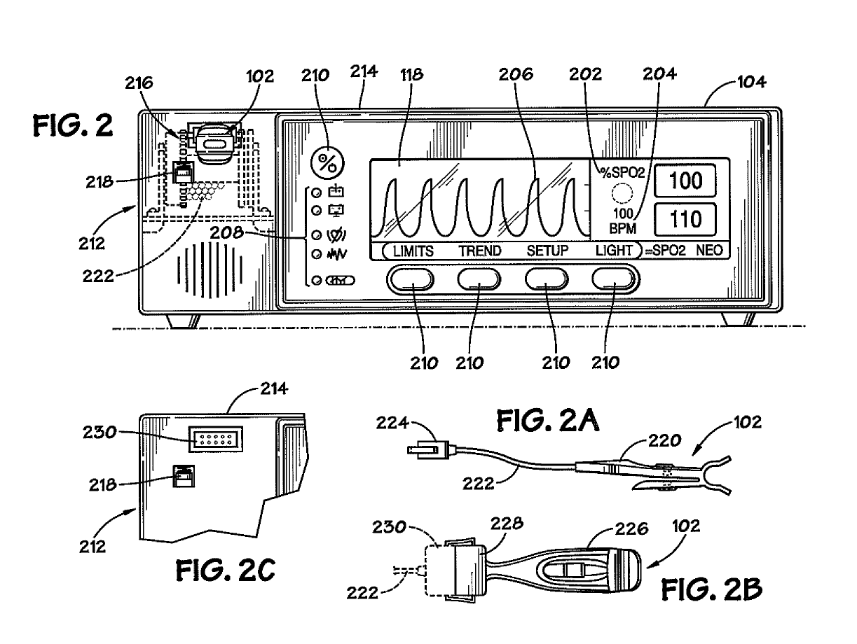

A front view of the sensor 102 and the monitor 104 described above is illustrated in FIG. 2, according to an embodiment. The monitor 104 may be configured to display the calculated parameters on a display 118. As illustrated in FIG. 2, the display 118 may be integrated into the monitor 104. However, in another embodiment, the monitor 104 may be configured to provide data via a port to a display (not shown) that is not integrated with the monitor 104. The display 118 may be configured to display computed physiological data including, for example, an oxygen saturation percentage 202, a pulse rate 204, and/or a plethysmographic waveform 206. As is known in the art, the displayed oxygen saturation percentage 202 may be a functional arterial hemoglobin oxygen saturation measurement in units of percentage SpO₂, while the displayed pulse rate 204 may indicate a patient's 108 pulse rate in beats per minute. The monitor 104 may also display information related to alarms, monitor settings, and/or signal quality via the indicator lights 208.

To facilitate user input, the monitor 104 may include a plurality of control inputs 210. The control inputs 210 may include fixed function keys, programmable function and/or soft keys, and soft keys. For example, the control inputs 210 may correspond to soft key icons in the display 118. Pressing control inputs 210 associated with, or adjacent to, an icon in the display may select a corresponding option.

The monitor 104 may also include a retraction housing 212 used to store a cable 222 that may be attached to the sensor 102. The retraction housing 212 may have a lid on the top most portion of the retraction housing 212. This lid may be used to gain access to the retraction mechanism 216 for cleaning or removal of the retraction mechanism 216. The lid may also allow for access to the cable 222 for cleaning, removal, or replacement. In one embodiment, the retraction housing 212 may be an integrated part of the monitor 104 and, thus, non-separable from casing 214. Alternatively, the retraction housing 212 may itself be removable from the monitor 104, and thus separable from the casing 214 used to enclose the monitor 104. In this manner, if the retraction mechanism 216 becomes damaged, repair or replacement of the damaged retraction mechanism 216 may accomplished separate from the monitor 104. In a further embodiment, the retraction housing 212 may itself be disposable, thus eliminating the need to clean or replace the retraction mechanism 216 or the cable 222.

In an embodiment, the retraction housing 212 may act to cover and protect the retraction mechanism 216 of the pulse oximeter. Similarly, the casing 214 may act to cover and protect the internal components of the monitor 104. The retraction housing 212 also may function to store a cable 222 when the sensor 102 is not in use. As illustrated, the sensor 102 is in the stored position with the cable and adapter inside of the retraction housing 212. When monitoring of a patient 108 is required, a user may be able to extend the sensor 102 from the retraction housing 212 by grasping and pulling on the sensor 102, thus extending the sensor 102 from the retraction housing 212. Once monitoring of a patient 108 is complete, depression of a retraction activation device 218 may cause the cable 222 attached to the sensor 102 to retract into the retraction housing 212.

The sensor 102, as described above, is illustrated in FIG. 2A. The sensor 102 may include a body 220 and a plug 224, which may be attached to the body 220 by a short cable 222, for example, of length 12 inches or less. In another embodiment, the body 220 may be integrated with the plug 224. The body 220 may include the emitter 106, the detector 110. The body 222, or alternatively the plug 224, may also include the encoder 130. Furthermore, the body 220 may be sized to contact the finger of a patient 108, as well as any other suitable tissue site. In this embodiment, the body 220 may include clips for ease of placement onto a patient 108.

An embodiment of the sensor 102 is illustrated in FIG. 2B. FIG. 2B shows a sensor 102 including an integrated body 226. The integrated body 226 is similar in function to the body 220 described above, however, the integrated body 226 is directly and physically coupled to the integrated plug 228 without a cable 222 disposed between the integrated body 226 and the integrated plug 228. The integrated plug 228 may perform in a similar manner to the plug 224. The integrated plug 228 may also be coupled to a sensor port 230. The sensor port 230 may act to connect the integrated plug 228 to the cable 222, which may be wound around the retraction mechanism 216. In this manner, the sensor port 230 may pass signals to and from the integrated sensor 102 and the monitor 104, by way of the cable 222.

According to an embodiment, FIG. 2C illustrates a retraction housing 212 portion of a pulse oximeter 100, which may be used in accordance with the sensor 102 of FIG. 2A or 2B. As illustrated, the cable 222 is fully retracted into the retraction housing 212 so that only the sensor port 230 may be seen. Furthermore, the integrated body 226 and the integrated adapter 228 have been removed from sensor port 230. The sensor port 230 may extend outwardly from the casing 214. This may allow a user to grasp the sensor port 230 to extend the cable 222 from the retraction housing 212.

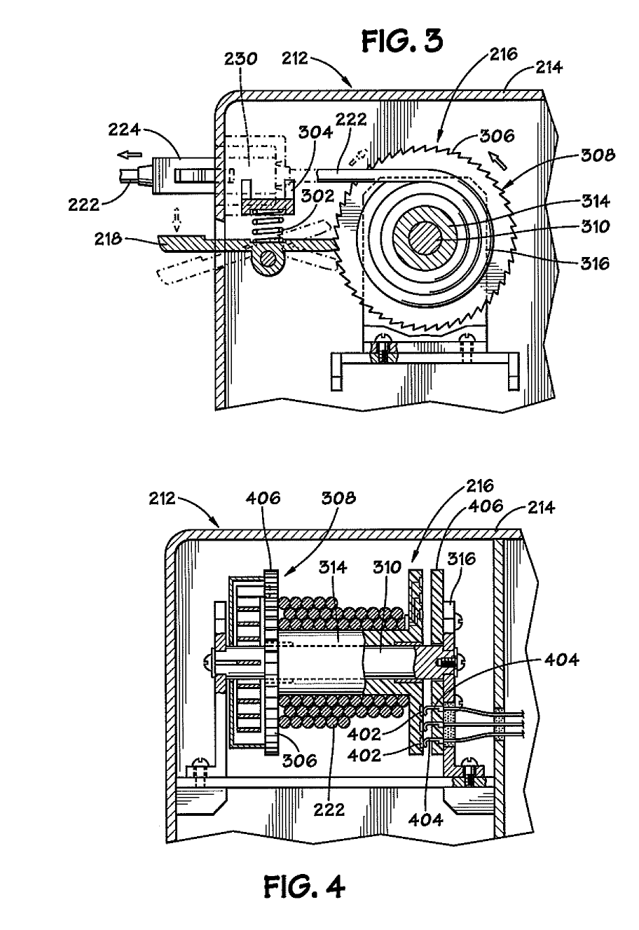

FIG. 3 illustrates a side view of the retraction housing 212 as well as the retraction mechanism 216 used in the storage of the cable 222, according to an embodiment. The retraction mechanism may include a spool 308, which may be used to aid in the storage of the cable 222. The spool 308 may be mounted on a support member 310. The support member 310 may pass through the spool 308 in such a manner as to allow rotational movement of the spool 308, while restricting lateral movement of the spool 308. The support member 310 may be attached to a support bracket 316. The spool 308 may also include an inner cylindrical member 314 with outer rims (not pictured). The inner cylindrical member 314 may be smaller in diameter than the rims, and may also reside between the rims. The cable 222 may be stored inside of the retraction housing 212 by being wrapped around the inner cylindrical member 314.

The retraction mechanism 216 may further include a tension spring (not pictured). The tension spring may be connected to the spool 308 and may cause rotation of the spool 308 in a first direction, for example clockwise. This clockwise rotation may cause the cable 222 to be wound around the inner cylindrical member 314. To prevent rotation of the spool 308 in this direction, a retraction activation device 218 may be used.

The retraction activation device 218 may extend inwards from the face of the retraction housing 212 and may be sized to contact the teeth 306 on at least one rim of the spool 308. The teeth 306 may be triangular in shape and may be aligned on an outer surface of the spool 308. The teeth 306 may further be aligned to allow the retraction activation device 218 to freely move along the edge of the spool 308 as the spool 308 rotates in one direction (for example counter-clockwise rotation). This alignment of the teeth allows a user to pull the cable 222 from the retraction housing 212 without using the retraction activation device 218. The alignment of the teeth 306 may also act to contact the retraction activation device 218 to prevent the spool 308 from rotating in a second direction (for example clockwise) when the user ceases to pull the cable 222 from the retraction housing 212. In this manner, the retraction activation device 218 may counteract the force of the tension spring to keep the cable 222 at a desired length when in use.

In an embodiment, the retraction activation device 218 may be a depressible tab connected to a lever. In another embodiment, the retraction activation device 218 may be a button. Regardless of the implementation of the retraction activation device 218, the function of the retraction activation device 218 is to allow or prevent the tension spring to cause the spool 308 to wind the cable 222 into the retraction housing 212. The retraction activation device 218 may be held in a first position by a resistance device 302, such as a spring. The resistance device 302 may be coupled to a base 304, which may be coupled to the inner wall of the retraction housing 212 for support. The resistance device 302 may act to provide a force upon the retraction activation device 218, which may act to resist movement of the retraction activation device 218 by keeping the retraction activation device 218 engaged with the teeth 306 of the spool 308 in the first position described above, thus counteracting the force of the tension spring.

When the sensor 102 is no longer in use, the user may depress the retraction activation device 218. Depressing the retraction activation device 218 causes the arm of the retraction device 218 to move to a second position where the retraction device ceases to engage the teeth 306 of the spool 308, allowing the tension spring to cause rotation of the spool 308 (for example, in a clockwise manner) to wind the cable 222 around the inner cylindrical member 314. This allows for storage of the cable 222 in the retraction housing 212.

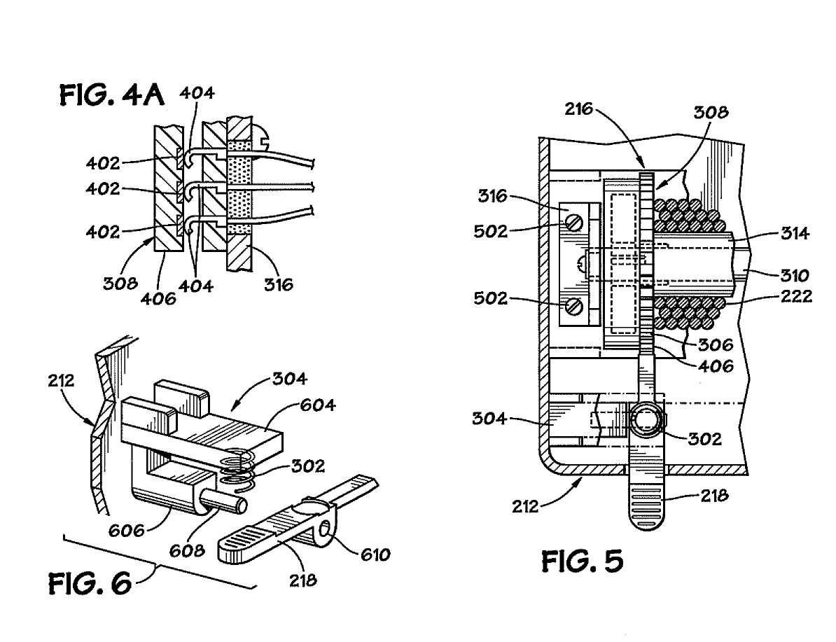

For illustrative purposes, a front view of the retraction mechanism 216 spool 308 including the spool 308 is shown in FIG. 4, according to an embodiment. As can be seen, the spool 308 includes two rims 406 that surround the inner cylindrical member 314. The teeth 306 may reside on either one or on both rims 406. The spool 308 is shown as being mounted on support member 310, which allows for rotational motion of the spool 308. The support member 310 may be attached to the support bracket 316 by a fastener, such as a screw, on either one or both ends of the support member. Furthermore, the cable 222 is shown as wound around the inner spool 314, and may terminate at the inner cylindrical member 314 at an interface (not pictured). This interface may be used to electronically couple the cable 222 to a set of slip rings 402. The slip rings 402 may be one or more conductive circles mounted on one external side of the spool 308. The slip rings 402 may be electronically insulated from the spool 308 as well as from each other. The slip rings 402 may contact a set of slip ring connectors 404. The slip ring connectors 404 may be made of a conductive material and may be connected to the internal circuitry of the monitor 104. In this manner, the interface may be used to communicate the electronic signals generated from the sensor 102 during monitoring of a patient 108 to the monitor 104 for processing and display.

FIG. 4A illustrates a close-up view of the slip rings 402 and the slip ring connectors 404, according to an embodiment. As can be seen, the slip rings 402 may be mounted directly into the spool 308. The spool 308 may be made of a non-conducting material such as hard rubber or plastic. In this manner, the slip rings 402 are insulated. The slip rings 402 also may contact the slip ring connectors 404. The slip ring connectors 404 may be mounted in the support bracket 316 and may extend outwards from the support bracket 316. In this manner, the spool 308 may be free to rotate in a circular manner, while never losing contact with the slip ring connectors 404. The slip ring connectors 404 may further be electronically coupled to the monitor 104, and as such, may complete a path to provide electronic signals to and from the sensor 102 and the monitor 104.

A top view of the retraction mechanism 216 is illustrated in FIG. 5, according to an embodiment. As can be seen, the retraction activation device 218 extends outwardly from the retraction housing 212. The resistance device 302 is shown as providing force to the retraction activation device 218 and is anchored by the base 304. Furthermore, the retraction activation device is shown as contacting the teeth 306 located on the rim 406. Also illustrated is the cable 222 wrapped around the inner spool 314. The spool 308 may be anchored to the bracket 316 by the support member 310 in such a manner as to allow rotational movement of the spool 308, while restricting lateral movement of the spool 308. The bracket 316 may be held in position by one or more fasteners 502, such as screws, which may be used to anchor the bracket 316 to the retraction housing 212.

FIG. 6 illustrates a detailed view of a support structure 602 for the retraction activation device 218, according to an embodiment. The support structure 602 may include the base 304. The base 304 may be fastened to the retraction housing 212. The base 304 may include a top portion 604 against which the resistance device 302 may contact. The base may also include a bottom portion 606 which may include a support probe 608. The support probe 608 may be sized to mate with the retraction activation device 218. The retraction activation device 218 may have an opening 610 into which the support probe 608 may fit. The opening 610 and support probe 608 may allow for vertical motion of the retraction activation device 218, while restricting lateral motion of the retraction activation device 218. In one embodiment, the support probe 608 may include a protrusion (not shown), which may be sized to mate with the opening 610 to restrict lateral movement of the retraction activation device 218.

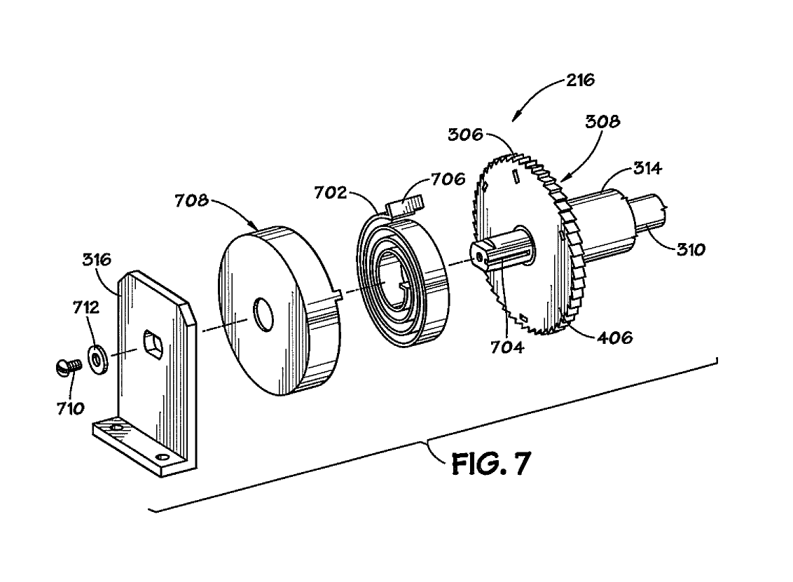

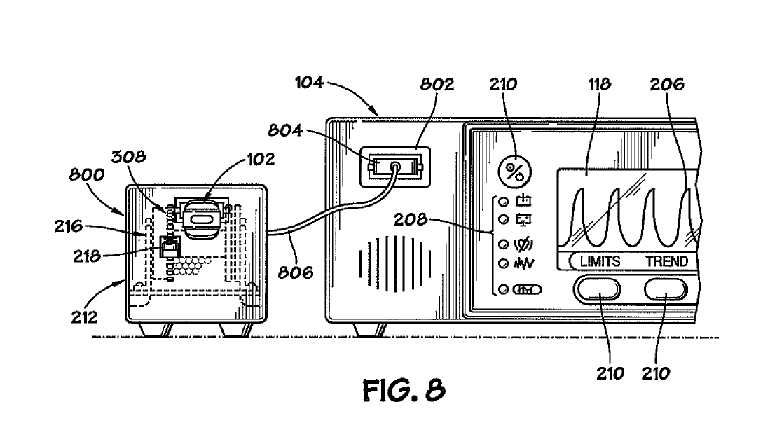

FIG. 7 shows an exploded view of the retraction mechanism in accordance with an embodiment. Illustrated is the spool 308 with the teeth 306 on the rim 406, inner cylindrical member 314, the support member 310, and the bracket 316 as described above. A fastener 710, which may be used to attach the support member 310 to the bracket 316, is also shown. Furthermore, a spacer 712, such as a washer, may be used to provide a buffer between the fastener 710 and the bracket 316.

Also illustrated is a torsion device 702. The torsion device 702 may be a torsion spring. The torsion device 702 may be a flexible elastic object made from, for example, a wire, a ribbon, or a bar of metal or rubber. The torsion device 702 may store mechanical energy when it is tightened, whereby the amount of torque it exerts is proportional to the amount it is tightened. The torsion device 702, as illustrated, may be coupled to the support member 310 in slot 704. Slot 704 may keep the torsion device 702 in a fixed position at one end, thus allowing the torsion device 702 to be tightened. The torsion device 702 may also include a flap 706. This flap 706 may contact a housing 708 and may be held in place by a fastener, or by any other means of fixing the flap 706 to the housing 708. In this manner, as the spool 308 rotates in one direction, for example as a user pulls on the cable 222 attached the spool 308, the torsion device 702 is tightened as energy is stored in the torsion device 702. This stored energy may not be enough to overcome the force applied by the engaged retraction activation device 218 contacting the teeth 306 of the spool 308. However, when the retraction activation device 218 is disengaged from the teeth 306, the spool 308 is no longer restricted and the torsion device 702 may act to loosen, which causes the spool 308 to rotate, which, in turn, winds the cable 222 around the inner cylindrical member 314. In this manner the cable 222 may be automatically retracted into the retraction housing 312.

FIG. 8 illustrates an embodiment illustrating a pulse oximeter system with a retraction device 800 attached to a monitor 104. As in with the pulse oximeter system described in FIG. 2, the monitor 104 includes a display 118 that may be configured to display calculated parameters of a patient, such as a plethysmographic waveform 206. The monitor also may display information related to alarms, monitor settings, and/or signal quality via the indicator lights 208, The monitor 104 may further include a plurality of control inputs 210, such as fixed function keys, programmable function keys, and soft keys. The monitor may also include a sensor port 802. The sensor port 802 may be used to connect an adapter 804 to the monitor 104. The adapter 804 is connected to the cable 806 and may function with the cable 806 to transmit and receive signals with the retraction device 800.

The retraction device 800 may include a retraction housing 212 and a retraction mechanism 216. The retraction mechanism 216, as described above, may operate to retract a cable from the sensor 102 into the retraction housing 212 using a spool 308. In this embodiment, the retraction housing 212 is separate from the monitor 104. Indeed, when the adapter 804 is removed from the monitor 104, there ceases to be a connection between the retraction device 800 and the monitor 104. This allows for easy cleaning, storage, or disposal of the retraction device 800.

Specific embodiments have been shown by way of example in the drawings and have been described in detail herein. However, it should be understood that the claims are not intended to be limited to the particular forms disclosed. Rather, the claims are to cover all modifications, equivalents, and alternatives falling within their spirit and scope.

What is claimed is:

1. A medical device comprising:
a monitor adapted obtain a physiologic signal from a patient; and
a retraction housing comprising a retraction mechanism adapted to retract a cable.

2. The medical device of claim 1, wherein the retraction mechanism comprises a spool having a first portion adapted to contact a retraction activation device and a second portion adapted to wind the cable.

3. The medical device of claim 2, wherein the first portion comprises a rim with teeth disposed on the rim.

4. The medical device of claim 3, wherein the teeth restrict rotation of the spool only in one direction when contacted with the retraction activation device.

5. The medical device of claim 3, wherein the spool rotates in response to a torsion device when the retraction activation device is not contacted with the teeth.

6. The medical device of claim 1, wherein the cable is adapted to connect to a sensor.

7. The medical device of claim 6, wherein the sensor is adapted to emit electromagnetic radiation into a tissue sample of the patient and detect scattered and reflected light from the tissue sample.

8. The medical device of claim 7, wherein the sensor is adapted to generate the physiologic signal corresponding to the scattered and reflected light detected and to direct the physiologic signal to the retraction housing.

9. The medical device of claim 8, wherein the retraction housing directs the physiologic signal to the monitor.

10. The medical device of claim 1, wherein the medical device comprises a pulse oximeter.

11. A sensor cable retraction apparatus, comprising:
a sensor adapted to obtain readings from a patient;
a retraction mechanism adapted to retract a sensor cable coupled to the sensor in response to the activation of a retraction activation device; and
a retraction housing adapted to store the retraction mechanism and the sensor cable.

12. The retraction apparatus of claim 1, wherein the retraction housing is integrated into a monitor.

13. The retraction apparatus of claim 11, wherein the retraction housing is separable from a monitor.

14. The retraction apparatus of claim 11, wherein the retraction housing is externally connected to a monitor.

15. A method of storing a sensor cable comprising:
activating a retraction activation device on a medical device, wherein the activating step disengages the retraction activation device from contact with a rim of a spool in the retraction device to allow the spool to wind a sensor cable around the spool.

16. The method of claim 15, wherein disengaging the retraction activation device from the rim allows for the release of stored energy in a torsion device coupled to the spool.

17. The method of claim 12, comprising deactivating the retraction activation device to stop rotation of the spool.

18. The method of claim 17, wherein the rotation of the spool is stopped by the retraction activation device contacting teeth on the rim of the spool.

19. The method of claim 15, wherein the sensor cable is coupled to a sensor adapted to adapted to emit electromagnetic radiation into a tissue sample of a patient, detect the scattered and reflected light from the tissue sample, generate a physiologic signal corresponding to the scattered and reflected light detected, and to direct the physiologic signal to the medical device.

20. The method of claim 19, wherein the medical device is a pulse oximeter.
